(19) United States
(12) Patent Application Publication
(10) Pub. No.: US 2008/0300502 A1
(43) Pub. Date: Dec. 4, 2008

(54) Method Of Correlating Internal Tissue Movement

(75) Inventors: Guang-Zhong Yang, Surrey (GB); Nicholas Ablitt, London (GB); David N. Firmin, Surrey (GB)

Correspondence Address: HICKMAN PALERMO TRUONG & BECKER, LLP, 2055 GATEWAY PLACE, SUITE 550, SAN JOSE, CA, 95110, US

(73) Assignee: IMPERIAL INNOVATIONS LIMITED, London (GB)

(21) Appl. No.: 11/630,717

(22) PCT Filed: Jun. 23, 2005
(86) PCT No.: PCT/GB2005/002493

§ 371 Date: Jul. 11, 2008

(30) Foreign Application Priority Data
Jun. 25, 2004 (GB) 0414328.5

(51) Int. Cl. A61B 5/113

(52) U.S. Cl. 600/534

(57) ABSTRACT

Internal tissue movement is correlated with the external body surface movement by tracking external surface movement at a plurality of surface point (12), simultaneously imaging internal tissue movement and correlating the external and internal movement using partial least squares regression to obtain a correlation model. In subsequent techniques, internal tissue movement can be predicted from measured external surface movement using the correlation model.

The invention relates to a method of correlating internal tissue movement for example for deriving respiratory induced cardiac deformation.

A significant problem with existing tissue imaging techniques for example in a human patient arises from involuntary acyclic motion. Such motion can be induced by the patient breathing which can compromise the imaging techniques because of the resultant movement or deformation of the tissue being imaged.

Various cardiac imaging techniques are known including Positron Emission Tomography (PET), 3D Echo Cardiography and Cardiovascular Magnetic Resonance (MR) techniques and in all of these respiratory induced cardiac deformation is a significant and limiting factor especially at high resolutions when it is desired to image vessel walls and coronary arteries. Cross-modal imaging techniques can give rise to difficulties because of incompatibilities with the respective apparatuses required—for example cardiovascular MR imaging can be compromised if additional metallic objects are in the vicinity.

Various solutions have been proposed for the problem of respiratory induced cardiac deformation and its impairment of imaging techniques. One solution is to require the patient to suspend breathing but this can only be for a limited duration, can induce stress in the patient which itself can affect the readings taken and indeed is not always possible, for example with an unconscious patient.

Alternatively respiratory gating is used. According to this technique the patient's breathing pattern is monitored and data is filtered so as to exclude data during breathing movement. One particular approach incorporates a navigator echo in which a column of material perpendicular to the respiratory motion has a read-out gradient giving its position allowing a decision to be made on which data should be retained. This technique can be incorporated, for example, with cardiovascular MR as discussed in Ehman R L, Felmlee J P. “Adaptive technique for high-definition MR imaging of moving structures”, Radiology. 1989;173(1):255-263.

A further proposed solution is to obtain a measure of movement of the patient's chest by measuring its expansion. This is achieved by strapping a bellows-type arrangement around the user's chest and measuring the movement of or strain on a point on the bellows. However a problem with this approach is that the surface distortion is poorly coupled to the induced cardiac motion such that the technique is highly inaccurate.

The invention is set out in the claims. In particular monitoring movement of the surface (chest) movement at multiple points provides additional data and the use of a regression technique, for example Partial Least Squares Regression (PLSR) to correlate the surface movement with the internal tissue movement, ensures that a good correlation is achieved. Indeed the preferable use of PLSR effectively resolves the problem encountered by traditional regression methods in that the latent variables from both the input and output of the regression model are used to establish inner relationships.

Embodiments of the invention will now be described, by way of example, with reference to the drawings, of which:

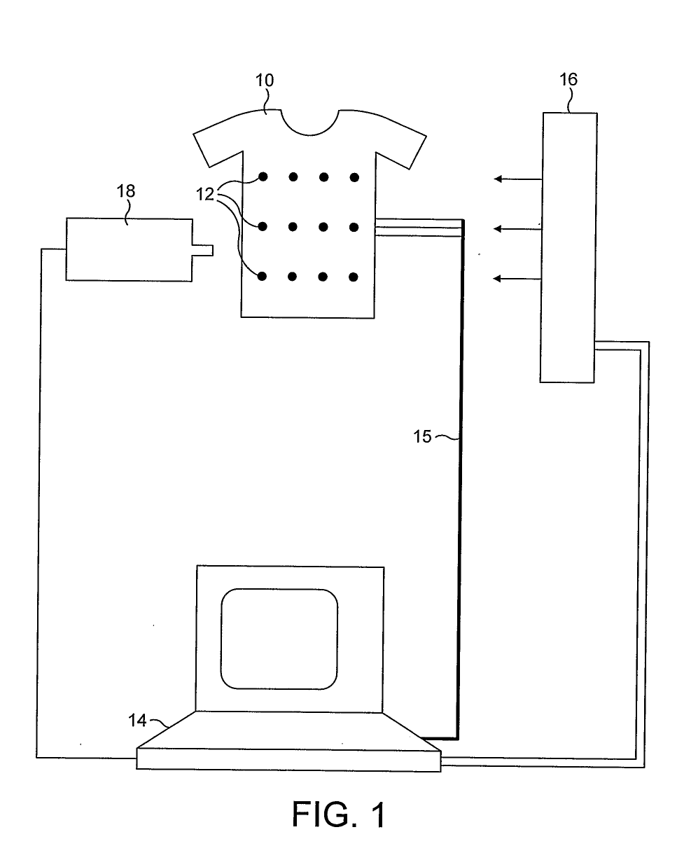

FIG. 1 is a diagramatic representation of an apparatus according to the invention;

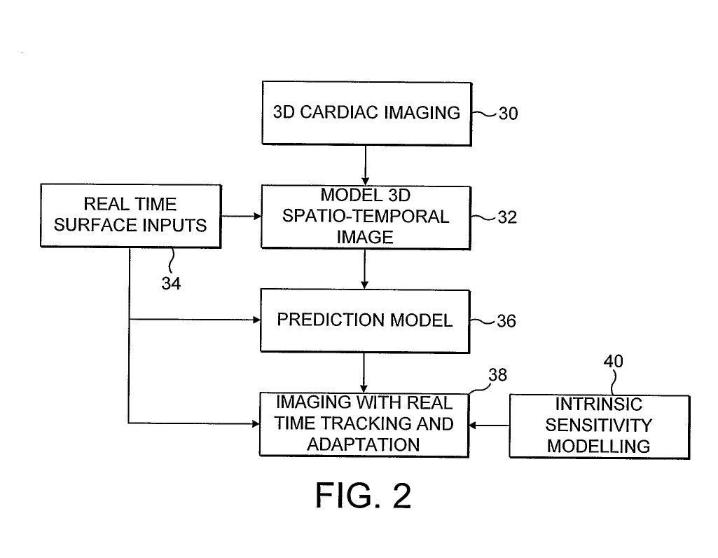

FIG. 2 is a flow diagram showing operation of the invention; and

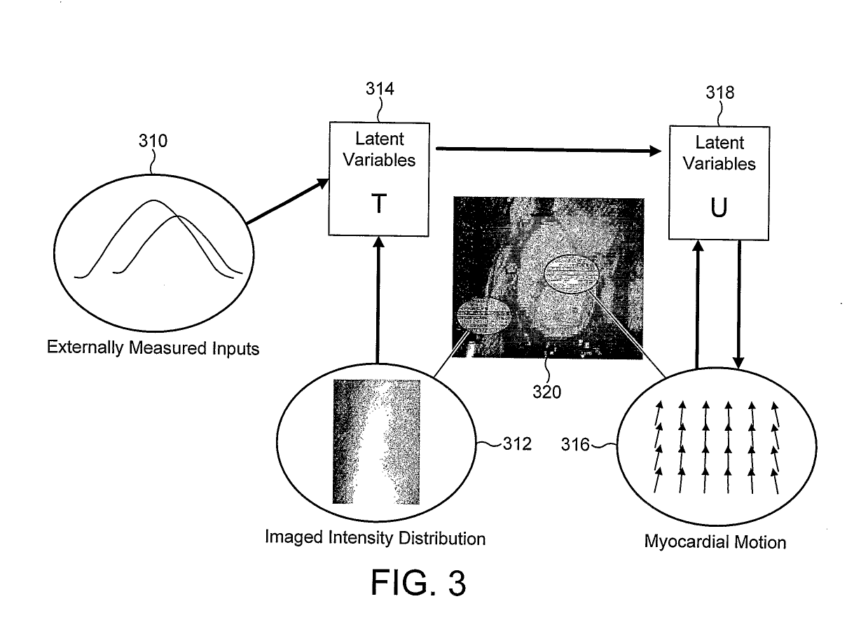

FIG. 3 is a diagram showing implementation of the method.

In overview, the method according to the invention correlates simultaneous measurements of three dimensional heart movement and two dimensional chest surface (wall) movement. A relationship between these two factors is then extracted using partial least squares regression (PLSR) to provide a mapping of two dimensional chest wall movements to predicted three dimensional heart movement. The correlation model hence obtained is derived in a calibration stage on a patient. As a result in a subsequent prediction phase, easily measurable 2D chest surface movement can be obtained and 3D cardiac motion predicted using the mapping allowing tracking of movement of the internal anatomical region of interest. This allows, for example, operations or treatments such as radiotherapy to take place incorporating compensation for heart movement without the need for complex or incompatible heart imaging, but just using the simultaneous measured 2D surface movement which can be obtained, for example, from a tension jacket on the patient. The approach is also useful for improving cardiac imaging generally.

An apparatus appropriate for carrying out the technique is shown in FIG. 1. A two dimensional chest surface measurement tension jacket 10 detects displacement at the chest surface at a plurality of points 12 and outputs the displacement data to a processor 14 via a bus 15. A cardiovascular MR array 16 simultaneously obtains a dynamic 3D MR image of the heart and outputs the image to processor 14. Processor 14 constructs the image of the spatio-temporal deformation of the heart and correlates the movement to the measured 2D chest surface movements using PLSR during the calibration phase.

In the subsequent prediction phase the wearer of the jacket 10 undergoes a procedure such as a radiotherapy operation in which radiotherapy is carried out by an apparatus as shown generally at 18. The processor 14 controls the radiotherapy beam dependent on respiratory induced cardiac deformation for example in order to avoid irradiating cardiac tissue temporarily obscuring the area on which therapy is being carried out. The cardiac deformation is predicted or modelled by the processor 14 based on the 2D surface measurements simultaneously obtained from the tension jacket 10, using the correlation mapping obtained during the calibration phase. Optimally the calibration and prediction phases are carried out immediately one after the other.

Alternatively the prediction phase can be used to remove blurring of 3D imaging due to respiratory motion. In this case, subsequent to the training phase, but whilst wearing the tension jacket the patient undergoes further 3D scanning which may be the same or a different modality than that used to capture the 3D information during the training phase. In this case the captured 3D images can be corrected using the predicted 3D motion derived from the readings from the tension jacket. As a result the approach can be implemented for motion checking during imaging or therapy to compensate for motion-induced artefacts and degradation such as respiratory induced blurring.

The invention can be further understood with respect to the flow diagram shown in FIG. 2. At block 30 the three dimensional imaging step is carried out using cardiovascular MR. At block 32 modelling of the imaged data and registration to a selected reference volume is carried out to obtain a three dimensional spatio-temporal image effectively reflecting the respiration induced deformation of the heart overtime against the selected reference volume. The modelled image is correlated with real time measured surface inputs at block 34 and a prediction model is derived from the correlation at block 36. At block 38, subsequent to the modelling/prediction/calibration phase, real time measured surface inputs from block 34 are input to block 38 to provide imaging with real time tracking and adaptation for cardiac movement. At block 40 intrinsic motion sensitivity to modelling of the imaging process is carried out and input to imaging block 38 allowing adjustment of scanning parameters on the fly depending on the information derived from the motor modelling.

The tension jacket 10 shown in FIG. 1 can be any appropriate garment incorporating multiple strain and/or curvature or bend sensors as will be well known to the skilled reader, for example optical, ultrasonic, tension or pressure sensors which are compatible with the 3D imaging modality. Alternatively multiple optically readable points whose displacement can be measured by a remote sensor for example of the type manufactured under the name “NDI Polaris” by Northern Digital Inc of Ontario, Canada can be used. Such a sensor can use infrared light to avoid interference from, for example, bright surgical lights. The optically readable points can for example be in the form of barcodes allowing additional data to be derived. Of course any surface movement tracking arrangement can be adopted. In a further alternative optically readable indicia can be painted or adhered or otherwise formed directly on the patient's skin, or displacement or strain sensors can be provided on a belt or array worn by the patient. In all cases, the sensed data provides a direct reading of the displacement of each point on the chest surface of the patient which is particularly advantageous as the data can be used with minimal processing as a representation of the chest movement during both the calibration and subsequent prediction phases.

In one embodiment an optical fibre sensor may be used for motion and/or curvature measurement. Such a sensor is described in “Evaluation of a novel plastic optical fibre sensor for axial strain and bend measurements”, K S C Kuang. W K Cantwell, and P J Scully, Meas. Sci. Technol. 13 (2202) 1523-1534, incorporated herein by reference.

Briefly, such a sensor includes one or more optical fibres, for example a plastic optical fibre, with a light source at one end and a detector at the other end. The fibre includes a portion of pre-determined lengths in which a segment of the cross section of the fibre is removed, for example by abraiding the surface of the fibre with a razor blade. When the fibre is straight, a certain amount of light will escape from the abraided portion. When the fibre is bent, such that the abraided portion is on the concave side of the fibre, the amount light escaping from the portion is reduced and the bend can detected by an increase in intensity of the light detected at the detector. Conversely, if the abraided portion is on the convex side of the bend, the bend in this direction will be detected as a decrease of the intensity of detected light because more light now escapes from the abraided portion.

The optical fibre sensors may be used in short length at the plurality of points 12. Alternatively, long fibres may be incorporated from one side of the chest to the other and from top to bottom of the chest such that global curvature of the chest can be detected.

The MR scanner 16 can be any appropriate scanner for example a Siemens Sonata MR scanner available from Siemens, Germany. Any other appropriate cardiac scanning/imaging device can alternatively be used. Similarly any appropriate processor 14 and supporting software can be adopted to implement the PLSR correlation approach described in more detail below.

The imaging and modelling techniques required to provide a 3D spatio temporal image of cardiac deformation will now be described in more detail.

To recover cardiac deformation and establish its intrinsic correlation with real time measurable surface signals, 3D image volumes depicting different stages of the cardiac deformation due to respiration are used. The extraction of 3D deformation vectors described above in relation to FIG. 2, block 32 is performed using the free-form image registration method. There are a range of free-form registration methods that have been used in medical imaging, and they can all be applicable to the current invention as a means of defining tissue deformation. In the present embodiment, Free-Form Deformation or FFD proposed by Rueckert D, Sonoda L I, Hayes C, Hill D L, Leach M L, Hawkes D J. Nonrigid registration using free-form deformations: application to breast MR images. IEEE Trans Med Imaging. 1999; 18(8): 712-721 is used. With this technique, a hierarchical transformation model of soft tissue deformation is employed, in which the global motion of the heart is modelled by an affine transformation whereas local deformation is described by free-form deformation based on B-splines applied to a volumetric mesh of control points overlaid on the 3D image. Normalised Mutual Information (Studholme C, Hawkes D J, Hill D L G, A Normalised Entropy Measure of 3D Medical Image Analysis. Proceedings of SPIE Medical Imaging, San Diego Calif., February 1998; 3338:132-142) is used as a voxel-based similarity measure and registration is achieved by minimising a cost function that encapsulates contributions associated with both the smoothness of the transformation and the overall image similarity.

To ensure a good optimisation performance, the algorithm works by decoupling global and local motion such that only the affine transformation parameters are optimised initially. This is then followed by optimising the non-affine transformation parameters at increasing levels of resolution of the control point mesh. In the present embodiment, the final number of control points used is 9×9×9 to cover the image volume, which gives the total degrees-of-freedom of 2187. With this registration approach, the deformation of each volume in relation to the selected reference volume (in relation to which registration has taken place) is characterised by the movement of control vertices of the B-splines. The associated 729 3D vectors are then used for correlation with the chest surface movement measurements. When other registration techniques are used, the dimensionality of the motion vector will be dictated by the deformation parameters. As discussed below a PLSR algorithm is used to determine the intrinsic relationship with real-time measurable signals associated with different levels of respiratory motion. Of course, other methods such as nonlinear regression techniques, for example kernel bases PLSR can also be used.

The PLSR technique used to correlate the 3D heart data with the 2D chest surface data will be generally well known to the skilled reader and the basic technique is described in Wold, H. “Soft modelling with latent variables: the nonlinear iterative partial least squares approach”. Perspectives in probability and Statistics: Papers in honour of M. S. Barlett, (J Gani, ed). London: Academic Press. 1975: 114-142. A particular benefit of PLSR is that it is designed to extract intrinsic relationships between data sets. Its ability to extract correlations between input and output data that is itself highly collinear, allows it to deal with problems that would be inappropriate for multi linear or principal components regression. For completeness a treatment of the implementation of PLSR to obtain the correlation model of the present invention will now be described.

If we assume X as being the surface measurements at a given instant of the respiratory cycle (predictor) and Y being the respiratory induced cardiac motion (response), there will be a significant amount of redundancies in both X and Y. This is because the FFD model used for deformation recovery in Y involves uniformly sampled control vertices and some of the vertices may be strongly correlated depending on the cardiac structure being covered. Conversely, the placement of surface measurements for real-time monitoring of respiratory motion is difficult to control and redundancies are inevitable.

In this case, the commonly used multivariate regression methods such as principal components regression or canonical regression are not suitable. This is because for these techniques, factors underlying the response (Y) and predictor (X) variables are extracted from either the Y^{T}Y or the X^{T}X matrices. They also have the restriction that the number of prediction functions can not exceed the minimum number of X and Y variables. As a result these techniques may pick out the most significant variations in X and Y individually but not necessarily those most significant for determining the relationship between X and Y.

By contrast, PLSR regression finds components from X that are also relevant for Y. Specifically, PLSR searches for a set of components called latent vectors that performs a simultaneous decomposition of X and Y with the constraint that these components explain as much as possible of the covariance between X and Y. In practice, it is possible that significant information for describing the variation in Y may be hidden in X to the extent that other techniques such as Principal Components Regression (PCR) may exclude this information as noise. In PLSR the direction in the space of X is sought, which yields the biggest covariance between X and Y. The method examines both the X and Y data and extracts the factors that are significant to both of them. The factors extracted are in order of significance, by evaluating X^{T}Y, to obtain the primary factor with which X determines the variation in Y.

Accordingly, applying PLSR, and assuming that the dimension used to describe the distribution of myocardial deformation (response) is q (for example 729 in the case of a 9×9×9 grid of control points) and the dimension used to describe each surface measurement for the respiratory motion is (predictor) p, when a total number of m experiments are performed to extract the relationship between X and Y, the size of the matrices will be m×p and m×q for X and Y, respectively. With PLSR, both the predictor and response matrices are decomposed, such that

X_{c}=TP^{T}+E (1)

and

Y_{c}=UQ^{T}+F (2)

Where T and U are latent variable between which PLSR seeks to find an inner relationship and E and F are factors in X and Y that are not described by the PLSR model T comprising a factor score matrix, P the factor loading matrix and Q the coefficient loading matrix. In the above equations, X_{c} and Y_{c} represent the mean centred matrices of X and Y, respectively. PLSR tries to find a score vector t (column of T) in the column space of X_{c} and a score vector u (column of U) in the column space of Y_{c} such that

t=X_{c}w (3)

u=Y_{c}q (4)

to give the maximal squared covariance for (u^{T}t)². That is, the process aims to maximise (q^{T}Y_{c}^{T}X_{c}w)² subject to |w|=|q|=1. It can be shown that from combining equations (3) and (4) the solution to this equation is given by an eigenvalue problem of X_{c}^{T}Y_{c}, i.e.,

X_{c}^{T}Y_{c}Y_{c}^{T}X_{c}w=λw (5)

where λ is the eigenvalue associated with w. In essence, the method searches for a set of latent vectors that performs a simultaneous decomposition of X and Y with the constraint that these components explain as much as possible of the covariance between X and Y. As can be seen from FIG. 3, rather than linking measurements X and Y directly, the method tries to establish the inner relationships between the latent variables T and U, derived from X and Y in equations (1) and (2) respectively.

In particular when imaging internal tissue 320, externally measured inputs (X) are received at 310 which can be imaged as the intensity distribution shown at 312. From the multiple measurements of surface intensity distributions the latent variable factors (T) 314 are extracted. A similar process is applied to the observed output data (Y), in this case the deformation vectors of the heart 316 derived from the control vertices of the three form image registration algorithm. From this the latent variables factor (U) 318 is extracted. In particular the following relationship is established.

U=TB+U_{E} (6)

where U_{E} is an error term similar to E and F above and B is a 1×1 diagonal matrix where the first 1 eigenvalues are used for prediction. When these error terms are ignored, we can obtain the predicted value of Y_{c} as

Y_{c}=TBQ^{T} (7)

where the values of B and Q are obtained from equation (1) to (6) and the value of T is obtained from the measured value of X_{c} and equation (1). In particular the solution to the equations can be solved iteratively through non-linear iterative partial least squares (NIPALS) techniques as described in Geladi P, Lowalski B. Partial least-squares regression: A tutorial. Analytic Chimica Acta, 1986; 185: 1-17. Of course any other appropriate approach to solving the equations, for Y_{c} can be adopted.

In order to acquire the cardiac image data, i.e. the response described in more detail above, 3D anatomical data of the target anatomy in response to motion needs to be acquired. This can be achieved by using any anatomical imaging techniques such as CT or MRI. In the present embodiment, this is achieved by using MR imaging which is carried out on a Siemens Sonata MR scanner having a field strength of 1.5 T, a peak gradient strength of 40 mT/m and a slew rate of 200 mT/ms. All images are acquired in the supine position and oversampled 3D datasets as discussed in Keegan J, Gatehouse P D, Yang G Z, Firmin D N. Coronary artery motion with the respiratory cycle during breath-holding and free-breathing: implications for slice-followed coronary artery imaging. Magn Reson Med. 2002; 47(3): 476-481. The duration of the examination is about 20 to 25 minutes, depending on the heart rate. The imaging parameters used include an EF flip angle of 65°, in plane matrix size of 256×102, pixel size of 1.56×2.70 mm, and field of view (FOV) of 400×275 mm. The 3D slab comprises 14 slices, covered by two segments with 51 views per segment. This gave a total of 28 segments per 3D slab. Data acquisition is repeated 20 times for a total acquisition duration of 560 cardiac cycles. Data is acquired with four receiver coils. All raw data, is stored and processed off-line. Images are then created from the raw data by using the 3D FFT. Contributions from all coils are combined with an equal weight. Image sets can be created for between six and seven different respiratory positions covering from end-inspiration to end expiration. In general, any MR pulse sequence that gives 3D coverage of the target anatomy at given motion position can be used for this invention.

The approach described herein provides numerous advantages. Cross modality reconstruction of patients specific models for dense motion field prediction are allowed which, after initial modelling, can be used in real-time prospective motion tracking or correction. As a result of the technique described above a large number of predictor variables can be used even when the principal modes of variation of the response (cardiac motion) variables are limited. The strength of the PLSR approach is that it additional permits reliable motion prediction when the number of observations is significantly less than the observed variables. Even though the surface intensity traces can be strongly coupled with each other but poorly correlated with respiratory induced cardiac deformation they can be used to accurately predict cardiac motion through the extraction of the latent variables of both the input and output of the model. It is particularly useful when the data involved is highly collinear as the approach accounts for redundancies in both the predictor (surface measurement) and response (cardiac motion). Furthermore the approach can be used to remove blurring due to respiratory motion.

It will be appreciated that elements of the embodiments described above can be interchanged and juxtaposed as appropriate and that the method steps can be carried out in any appropriate order.

It will be further appreciated that the approach can be applied to any organ, tissue or visceral/anatomical structure and can be used to correlate the motion of any appropriate part of a body surface. The technique can be used for any living matter such as humans or animals.

Any manner of obtaining movement data and correlating it can be adopted. For example registration based on free-form deformation (FFD) or finite element modelling (FEM) can be used to recover the underlying spatio-temporal deformation of the anatomical structure. To cater for non-linear relationships between respiratory traces and heart deformation non-linear and kernel based PLSR approaches may be used of the type described in Malthouse E, Tamhane A, Mah R. “Nonlinear partial least squares”. Computers in Chemical Engineering. 1997; 21(8): 875-890. The 3D motion prediction technique can be used on motion tracked imaging in MR as well as for other parallel imaging modalities such as PET, Computer Tomography (CT) or 3D Echo Cardiography and the delivery of focused imaging in the presence of physiological motion. Parallel imaging can be adopted to reduce imaging time. Although the use of surface tension arrays or optical approaches has been discussed, other techniques based on strain or surface position, or ultrasound based techniques can be used. Yet a further possibility is the use of micro-sensors. Alternatively chest intensity profiles can be used as a means of measuring local surface deformation. The techniques adopted are used within the constraints of modality compatability for example for MR in which the exclusion of ferromagnetic materials and the restriction of RF are of significant importance.

The techniques described can be used to support any appropriate application such as medical or diagnostic procedures in which the management of inconsistent physiological motion is required, such as motion tracking, calibration and detection.

1. A method of correlating internal tissue movement with external surface movement comprising tracking surface movement at a plurality of surface points, simultaneously obtaining tissue movement and correlating the surface movement to the tissue movement by a regression technique to obtain a correlation model.

2. A method as claimed in claim 1 in which the regression technique is partial least squares regression (PLSR).

3. A method as claimed in claim 1 in which the internal tissue movement comprises cardiac movement.

4. A method as claimed in claim 1 in which the surface movement is respiratory induced.

5. A method as claimed in claim 1 in which the surface movement is tracked using a tension jacket.

6. A method as claimed in claim 1 in which the surface movement is tracked remotely.

7. A method as claimed in claim 6 in which the surface movement is tracked optically.

8. A method as claimed in claim 1 in which the surface movement is tracked by measuring strain or curvature or both.

9. A method as claimed in claim 1 in which tissue movement is obtained using at least one of 3D magnetic resonance, Positron Emission Tomography, Computer Tomography or 3D Echo Cardiography.

10. A method of modeling internal tissue movement from surface movement comprising tracking surface movement at a plurality of surface points and modeling correlated internal tissue movement therefrom using a correlation model.

11. (canceled)

12. An internal tissue movement correlation apparatus comprising an external surface movement tracker reference having a plurality of trackable points.

13. An apparatus as claimed in claim 12 in which the reference comprises a garment on which the trackable points are provided.

14. An apparatus as claimed in claim 13 in which the trackable points comprise strain sensors.

15. An apparatus as claimed in claim 12 in which the trackable points are remotely trackable.

16. An apparatus as claimed in claim 12 in which the trackable points are optically trackable.

17. An apparatus as claimed in claim 12 in which the garment includes one or more optical fibres for sensing surface movement.

18. An apparatus as claimed in claim 17, the optical fibres being sensitised to detect bending thereof.

19. An apparatus as claimed in claim 18, the optical fibres including a longitudinal portion with a segment of its cross sectional profile being removed.

20. An apparatus as claimed in claim 12 further comprising a tracker apparatus arranged to track movement of the trackable points.

21. An apparatus as claimed in claim 12 further comprising an apparatus arranged to obtain movement of internal tissue.

22. An apparatus as claimed in claim 21 further comprising a processor arranged to process the obtained internal tissue movement and tracked external movement and derive a correlation model.

23. (canceled)

24. A method of correcting an internal tissue movement image using a related external surface movement comprising obtaining an imaged representation of the internal tissue movement, obtaining a predicted representation of the internal tissue movement from the external surface movement and correcting the imaged representation using the predicted representation.

25. A method as claimed in claim 24 in which the predicted representation comprises a model of internal tissue movement;
wherein the model of internal tissue movement is obtained by tracking surface movement at a plurality of surface points and modeling correlated internal tissue movement therefrom using a correlation model.

26.-28. (canceled)

29. A method as claimed in claim 10, wherein modeling correlated internal tissue movement using a correlation model comprises simultaneously obtaining tissue movement and correlating the surface movement to the tissue movement by a regression technique to obtain the correlation model.

30. A method as claimed in claim 24 in which the predicted representation comprises a model of internal tissue movement that correlates internal tissue movement with external surface movement by tracking surface movement at a plurality of surface points, simultaneously obtaining tissue movement and correlating the surface movement to the tissue movement by a regression technique to obtain a correlation model.

31. A computer-readable storage medium having recorded thereon a correlation model for correlating external surface movement with internal tissue movement in which the model comprises a mapping between movement of a plurality of surface points and internal tissue movement.

32. A computer-readable storage medium as claimed in claim 47, further comprising one or more instructions recorded thereon, wherein execution of the one or more instructions by one or more processors causes correlating internal tissue movement with external surface movement by tracking surface movement at a plurality of surface points, simultaneously obtaining tissue movement and correlating the surface movement to the tissue movement by a regression technique to obtain a correlation model.

33. A computer system, comprising:
one or more processors;
a computer-readable storage medium accessible to the one or more processors and having recorded thereon one or more instructions, wherein execution of the one or more instructions by one or more processors causes correlating internal tissue movement with external surface movement by tracking surface movement at a plurality of surface points, simultaneously obtaining tissue movement and correlating the surface movement to the tissue movement by a regression technique to obtain a correlation model.
